SEQUENCE LISTING (50 sequences)

SEQ ID NO: 1 - RNA, 23 nt, Artificial Sequence
  Synthetic
uccaccucaucauccacaaugau

SEQ ID NO: 2 - RNA, 21 nt, Artificial Sequence
  Synthetic
ucauuguggaugaugaggugg

SEQ ID NO: 3 - RNA, 22 nt, Artificial Sequence
  Synthetic
ccaccucaucauccacaaugau

SEQ ID NO: 4 - RNA, 20 nt, Artificial Sequence
  Synthetic
ucauuguggaugaugaggug

SEQ ID NO: 5 - RNA, 23 nt, Artificial Sequence
  Synthetic
ugguggagagucucacuuuccau

SEQ ID NO: 6 - RNA, 21 nt, Artificial Sequence
  Synthetic
uggaaagugagacucuccacc

SEQ ID NO: 7 - RNA, 22 nt, Artificial Sequence
  Synthetic
gguggagagucucacuuuccau

SEQ ID NO: 8 - RNA, 22 nt, Artificial Sequence
  Synthetic
uggaaagugagacucuccacca

SEQ ID NO: 9 - RNA, 23 nt, Artificial Sequence
  Synthetic
uaggugaccggguguacauacau

SEQ ID NO: 10 - RNA, 21 nt, Artificial Sequence
  Synthetic
  modified_base (14)..(14): N is Inosine
  misc_feature (14)..(14): n is a, c, g, or u
uguauguacacccngucaccu

SEQ ID NO: 11 - RNA, 23 nt, Artificial Sequence
  Synthetic
ugagacauccccuguggaugaau

SEQ ID NO: 12 - RNA, 21 nt, Artificial Sequence
  Synthetic
  modified_base (14)..(14): N is Inosine
  misc_feature (14)..(14): n is a, c, g, or u
uucauccacagggnaugucuc

SEQ ID NO: 13 - RNA, 23 nt, Artificial Sequence
  Synthetic
uacccuggccucucucuaucuau

SEQ ID NO: 14 - RNA, 21 nt, Artificial Sequence
  Synthetic
  modified_base (14)..(14): N is Inosine
  misc_feature (14)..(14): n is a, c, g, or u
uagauagagagagnccagggu

SEQ ID NO: 15 - RNA, 23 nt, Artificial Sequence
  Synthetic
ucacccugacuuucaacaccuau

SEQ ID NO: 16 - RNA, 21 nt, Artificial Sequence
  Synthetic
uagguguugaaagucagggug

SEQ ID NO: 17 - RNA, 23 nt, Artificial Sequence
  Synthetic
uccuuccaacacuggagugacau

SEQ ID NO: 18 - RNA, 21 nt, Artificial Sequence
  Synthetic
ugucacuccaguguuggaagg

SEQ ID NO: 19 - RNA, 23 nt, Artificial Sequence
  Synthetic
ucucaaguacccuucacugagau

SEQ ID NO: 20 - RNA, 21 nt, Artificial Sequence
  Synthetic
ucucagugaaggguacuugag

SEQ ID NO: 21 - RNA, 23 nt, Artificial Sequence
  Synthetic
ugguggagagucucacuuuccau

SEQ ID NO: 22 - RNA, 21 nt, Artificial Sequence
  Synthetic
uggaaagugagacucuccacc

SEQ ID NO: 23 - RNA, 23 nt, Artificial Sequence
  Synthetic
ugaaccgcccauuccuguuugau

SEQ ID NO: 24 - RNA, 21 nt, Artificial Sequence
  Synthetic
  modified_base (14)..(14): N is Inosine
  misc_feature (14)..(14): n is a, c, g, or u
ucaaacaggaaugngcgguuc

SEQ ID NO: 25 - RNA, 23 nt, Artificial Sequence
  Synthetic
ugcccauuccuguuugcuguguu

SEQ ID NO: 26 - RNA, 21 nt, Artificial Sequence
  Synthetic
  modified_base (14)..(14): N is Inosine
  misc_feature (14)..(14): n is a, c, g, or u
acacagcaaacagnaaugggc

SEQ ID NO: 27 - RNA, 23 nt, Artificial Sequence
  Synthetic
uccuguuuacuguguaugaucau

SEQ ID NO: 28 - RNA, 21 nt, Artificial Sequence
  Synthetic
ugaucauacacaguaaacagg

SEQ ID NO: 29 - RNA, 23 nt, Artificial Sequence
  Synthetic
uaagcagccguuucuccuugguu

SEQ ID NO: 30 - RNA, 21 nt, Artificial Sequence
  Synthetic
  modified_base (14)..(14): N is Inosine
  misc_feature (14)..(14): n is a, c, g, or u
accaaggagaaacngcugcuu

SEQ ID NO: 31 - RNA, 23 nt, Artificial Sequence
  Synthetic
ugcugcauagagugagcaguagu

SEQ ID NO: 32 - RNA, 21 nt, Artificial Sequence
  Synthetic
cuacugcucacucuaugcagc

SEQ ID NO: 33 - RNA, 23 nt, Artificial Sequence
  Synthetic
uuagcgcgagacuacuguuccau

SEQ ID NO: 34 - RNA, 21 nt, Artificial Sequence
  Synthetic
uggaacaguagucucgcgcua

SEQ ID NO: 35 - RNA, 23 nt, Artificial Sequence
  Synthetic
ucaguguucccuuuucaaguugu

SEQ ID NO: 36 - RNA, 21 nt, Artificial Sequence
  Synthetic
  modified_base (14)..(14): N is Inosine
  misc_feature (14)..(14): n is a, c, g, or u
caacuugaaaaggnaacacug

SEQ ID NO: 37 - RNA, 23 nt, Artificial Sequence
  Synthetic
uaguguucccuuuucaaguugau

SEQ ID NO: 38 - RNA, 21 nt, Artificial Sequence
  Synthetic
  modified_base (14)..(14): N is Inosine
  misc_feature (14)..(14): n is a, c, g, or u
ucaacuugaaaagngaacacu

SEQ ID NO: 39 - RNA, 23 nt, Artificial Sequence
  Synthetic
ucguguucccuuuucaaguugau

SEQ ID NO: 40 - RNA, 21 nt, Artificial Sequence
  Synthetic
  modified_base (14)..(14): N is Inosine
  misc_feature (14)..(14): n is a, c, g, or u
ucaacuugaaaagngaacacg

SEQ ID NO: 41 - RNA, 23 nt, Artificial Sequence
  Synthetic
uuugcauuaccuucgguuuguau

SEQ ID NO: 42 - RNA, 21 nt, Artificial Sequence
  Synthetic
uacaaaccgaagguaaugcaa

SEQ ID NO: 43 - RNA, 23 nt, Artificial Sequence
  Synthetic
ucugcauuaccuucgguuuguau

SEQ ID NO: 44 - RNA, 21 nt, Artificial Sequence
  Synthetic
uacaaaccgaagguaaugcag

SEQ ID NO: 45 - RNA, 23 nt, Artificial Sequence
  Synthetic
ucgccuucaguuuguauuuaguu

SEQ ID NO: 46 - RNA, 21 nt, Artificial Sequence
  Synthetic
acuaaauacaaacugaaggcg

SEQ ID NO: 47 - RNA, 23 nt, Artificial Sequence
  Synthetic
uugaccuccguguagugucuguu

SEQ ID NO: 48 - RNA, 21 nt, Artificial Sequence
  Synthetic
  modified_base (14)..(14): N is Inosine
  misc_feature (14)..(14): n is a, c, g, or u
acagacacuacacngagguca

SEQ ID NO: 49 - RNA, 23 nt, Artificial Sequence
  Synthetic
ucgaccuccguguagugucuguu

SEQ ID NO: 50 - RNA, 21 nt, Artificial Sequence
  Synthetic
  modified_base (14)..(14): N is Inosine
  misc_feature (14)..(14): n is a, c, g, or u
acagacacuacacngaggucg

(19) United States
(12) Patent Application Publication
(10) Pub. No.: US 2024/0191229 A1
(43) Pub. Date: Jun. 13, 2024

(54) RNA SILENCING AGENTS AND METHODS OF USE

(71) Applicant: ADARx Pharmaceuticals, Inc., San Diego, CA (US)

(72) Inventors: Zhen Li, San Diego, CA (US); Zhiqing (Joel) Zhou, San Diego, CA (US)

(73) Assignee: ADARx Pharmaceuticals, Inc., San Diego, CA (US)

(21) Appl. No.: 18/286,640

(22) PCT Filed: Apr. 13, 2022
(86) PCT No.: PCT/US2022/024692

§ 371 Date: Oct. 12, 2023

Related U.S. Application Data
(60) Provisional application No. 63/174,507, filed on Apr. 13, 2021.

(51) Int. Cl. C12N 15/113

(52) U.S. Cl. CPC C12N 15/113; C12N 2310/11; C12N 2310/315; C12N 2310/321; C12N 2310/322; C12N 2310/332; C12N 2310/344; C12N 2310/351; C12N 2310/533

(57) ABSTRACT

Aspects of the disclosure provide nucleic acids for reducing expression of a target RNA. In some aspects, the disclosure provides nucleic acid modifications and base-pairing configurations useful in the design of nucleic acids for RNA interference.

### CROSS-REFERENCE TO RELATED APPLICATIONS

This application claims priority under 35 U.S.C. § 119(e) to U.S. Provisional Patent Application No. 63/174,507, filed Apr. 13, 2021, which is hereby incorporated by reference in its entirety.

### REFERENCE TO A SEQUENCE LISTING SUBMITTED AS A TEXT FILE

The instant application contains a Sequence Listing which has been submitted in ASCII format and is hereby incorporated by reference in its entirety. Said ASCII copy, created on Apr. 12, 2022, is named A127870007WO00-SEQ-JIB and is 10,192 bytes in size.

### BACKGROUND

The field of RNA interference (RNAi) has received considerable interest in recent years, as RNA silencing agents provide the ability to knock down expression of a particular protein with a high degree of sequence specificity. RNAi has been useful in scientific research, for example, to study genetic and biochemical pathways, to elucidate the function of individual genes and gene products, and as a tool for target validation in the pharmaceutical industry. Additionally, substantial efforts are made with the goal of developing RNA silencing agents capable of mediating RNAi as a therapeutic strategy.

### SUMMARY

Among other aspects, the disclosure provides nucleic acid design strategies which can be useful in the design of RNA silencing agents. In some aspects, the disclosure relates to the discovery that an effective reduction in target RNA levels can be achieved using an antisense strand configured to mediate a wobble base-pairing between its position 14 nucleotide and the target RNA. Accordingly, in some aspects, the disclosure provides nucleic acids comprising an antisense strand having, at position 14 from its 5′ end, a nucleotide that forms a wobble base pair with a target nucleotide at a corresponding position on the target RNA.

In some aspects, the disclosure provides a nucleic acid for reducing expression of a target mRNA, the nucleic acid comprising an antisense strand of 15 to 31 nucleotides in length having a sequence that is at least 90% complementary to a contiguous sequence of the target mRNA, where the sequence of the antisense strand comprises, at position 14 from its 5′ end, an abasic site or a nucleotide that does not form a canonical (e.g., Watson-Crick) base pair with a target nucleotide at a corresponding position on the contiguous sequence of the target mRNA.

In some embodiments, the nucleotide at position 14 on the antisense strand and the target nucleotide at a corresponding position on the target mRNA are mismatched (e.g., the nucleotides form a mismatched base pair, such as a wobble base pair). In some embodiments, the mismatched base pair is a wobble base pair. For example, in some embodiments, the nucleotide at position 14 on the antisense strand forms a wobble base pair with the target nucleotide. In some embodiments, the target nucleotide comprises either cytidine or guanosine. In some embodiments, the nucleotide at position 14 on the antisense strand comprises either inosine or uridine. In some embodiments, the wobble base pair is I:C or U:G. In some embodiments, the nucleotide at position 14 on the antisense strand comprises inosine if the target nucleotide comprises cytidine. In some embodiments, the nucleotide at position 14 on the antisense strand comprises uridine if the target nucleotide comprises guanosine.

In some embodiments, the antisense strand comprises at least one modified nucleotide and/or at least one modified internucleotide linkage. In some embodiments, the antisense strand comprises one or more nucleoside modifications selected from 2′-aminoethyl, 2′-fluoro, 2′-O-methyl, 2′-O-methoxyethyl, and 2′-deoxy-2′-fluoro-β-d-arabinonucleic acid. Additional examples of nucleoside modifications are described elsewhere herein and include, without limitation, modified sugars, such as 2′-O substitutions to the sugar (e.g., ribose), including 2′-O-methoxyethyl sugar, a 2′-fluoro sugar modification (2′-fluoro), a 2′-O-methyl sugar (2′-O-methyl), 2′-O-ethyl sugar, 2′-Cl, 2′-SH, and substitutions thereof (e.g., 2′-SCH₃), a bicyclic sugar moiety, or substitutions such as a 2′-O moiety with a lower alkyl or substitutions thereof (e.g., —CH₃, —CF₃), 2′-amino or substitutions thereof, 2′,3′-seco nucleotide mimic, 2′-F-arabino nucleotide, inverted nucleotides, inverted 2′-O-methyl nucleotide, 2′-O-deoxy nucleotide, an alkenyl, an alkynyl, a methoxyethyl (2′-O-MOE), an —H (as in DNA), or other substituent. In some embodiments, the antisense strand comprises at least one phosphorothioate internucleotide linkage. In some embodiments, the sequence of the antisense strand, with the exception of the nucleotide that forms the wobble base pair, is 100% complementary to the contiguous sequence of the target mRNA.

In some embodiments, the antisense strand is 15 to 25 nucleotides in length (e.g., 15, 16, 17, 18, 19, 20, 21, 22, 23, 24, or 25 nucleotides in length). In some embodiments, the antisense strand is 19 to 25 nucleotides in length. In some embodiments, the antisense strand is 21 nucleotides in length. In some embodiments, the sequence of the antisense strand is at least 80% identical to a nucleotide sequence of Table 1. In some embodiments, the sequence of the antisense strand is at least 85% identical (e.g., at least 90% identical, at least 95% identical, or 100% identical) to a nucleotide sequence of Table 1. In some embodiments, the sequence of the antisense strand is at least 80% identical to any one of SEQ ID NOs: 2, 4, 6, 8, 10, 12, 14, 16, 18, 20, 22, 24, 26, 28, 30, 32, 34, 36, 38, 40, 42, 44, 46, 48, and 50. In some embodiments, the sequence of the antisense strand is at least 85% identical (e.g., at least 90% identical, at least 95% identical, or 100% identical) to any one of SEQ ID NOs: 2, 4, 6, 8, 10, 12, 14, 16, 18, 20, 22, 24, 26, 28, 30, 32, 34, 36, 38, 40, 42, 44, 46, 48, and 50.

In some embodiments, the nucleic acid further comprises a sense strand of 15 to 40 nucleotides in length (e.g., 15-35, 15-30, 15-25, 19-30, 19-25, or 25-30 nucleotides in length). In some embodiments, the sense strand forms a duplex region with the antisense strand. In some embodiments, the duplex region comprises a canonical or non-canonical base pairing between a nucleotide on the sense strand and the nucleotide at position 14 on the antisense strand. In some embodiments, the nucleotide on the sense strand comprises cytidine, adenosine, or uridine, if the nucleotide at position 14 on the antisense strand comprises inosine. In some embodiments, the nucleotide on the sense strand comprises adenosine if the nucleotide at position 14 on the antisense strand comprises uridine. In some embodiments, the sequence of the sense strand is at least 80% identical to any one of SEQ ID NOs: 1, 3, 5, 7, 9, 11, 13, 15, 17, 19, 21, 23, 25, 27, 29, 31, 33, 35, 37, 39, 41, 43, 45, 47, and 49. In some embodiments, the sequence of the sense strand is at least 85% identical (e.g., at least 90% identical, at least 95% identical, or 100% identical) to any one of SEQ ID NOs: 1, 3, 5, 7, 9, 11, 13, 15, 17, 19, 21, 23, 25, 27, 29, 31, 33, 35, 37, 39, 41, 43, 45, 47, and 49.

In some embodiments, the sense strand comprises at least one modified nucleotide and/or at least one modified internucleotide linkage. In some embodiments, the sense strand comprises one or more nucleoside modifications selected from 2′-aminoethyl, 2′-fluoro, 2′-O-methyl, 2′-O-methoxyethyl, and 2′-deoxy-2′-fluoro-β-d-arabinonucleic acid. Additional examples of nucleoside modifications and modified nucleotides are described elsewhere herein. In some embodiments, the sense strand comprises at least one phosphorothioate internucleotide linkage. In some embodiments, the sense strand is conjugated to at least one N-acetylgalactosamine (GalNAc) moiety.

In some aspects, the disclosure provides a nucleic acid for reducing expression of a target mRNA, the nucleic acid comprising an antisense strand of Formula (I):

5′-X⁰¹X⁰²X⁰³X⁰⁴X⁰⁵X⁰⁶X⁰⁷X⁰⁸X⁰⁹X¹⁰X¹¹X¹²X¹³X¹⁴(X^{Y})_{b}Nₐ-3′ (I),

where: each instance of N and X^{Y} is independently any type of nucleotide; a is an integer from 0-2, inclusive; b is an integer from 1-17, inclusive; X⁰¹-X¹³ are each independently any type of nucleotide, with the proviso that X⁰¹—(X^{Y})_{b} is at least 90% complementary to a contiguous nucleotide sequence of the target mRNA; and X¹⁴ is an abasic site or a nucleotide that does not form a canonical (e.g., Watson-Crick) base pair with a target nucleotide at a corresponding position on the contiguous nucleotide sequence of the target mRNA.

In some embodiments, “X” nucleotides of Formula (I) denote nucleotides forming a region of complementarity to a target mRNA as described elsewhere herein. In some embodiments, “N” nucleotides of Formula (I) denote optional nucleotides outside of the region of complementarity. In some embodiments, where the nucleic acid further comprises a sense strand in duplex with the antisense strand of Formula (I), “N” nucleotides denote optional nucleotides forming an overhang as described elsewhere herein.

In some embodiments, a is an integer from 1-2, inclusive. In some embodiments, a is 0. In some embodiments, b is an integer from 1-11, inclusive. In some embodiments, b is an integer from 5-11, inclusive. In some embodiments, b is 7. In some embodiments, the sequence of X⁰¹—(X^{Y})_{b} is at least 80% identical to a nucleotide sequence of Table 1. In some embodiments, the sequence of X⁰¹—(X^{Y})_{b} is at least 85% identical (e.g., at least 90% identical, at least 95% identical, or 100% identical) to a nucleotide sequence of Table 1.

In some embodiments, the sequence of X⁰¹—(X^{Y})_{b} is at least 95% complementary to a contiguous nucleotide sequence of a target mRNA. In some embodiments, the sequence of X⁰¹—(X^{Y})_{b} is 100% complementary to a naturally occurring contiguous nucleotide sequence of a target mRNA with the exception of X¹⁴, where: (i) X¹⁴ comprises inosine, and the target nucleotide at a corresponding position on the target mRNA comprises cytidine; or (ii) X¹⁴ comprises uridine, and the target nucleotide comprises guanosine. In some embodiments, b is 7, and the sequence of X⁰¹-X²¹ is 100% complementary to a naturally occurring contiguous nucleotide sequence of a target mRNA with the exception of X¹⁴, where: (i) X¹⁴ comprises inosine, and the target nucleotide comprises cytidine; or (ii) X¹⁴ comprises uridine, and the target nucleotide comprises guanosine. In some embodiments, the sequence of X⁰¹—(X^{Y})_{b} is 100% complementary to a naturally occurring contiguous nucleotide sequence of a target mRNA with the exception of X¹⁴ as described previously, and with the exception of X⁰¹, where X⁰¹ and a nucleotide at a corresponding position on the target mRNA comprise a mismatched base pair.

In some embodiments, the target nucleotide comprises either cytidine or guanosine. In some embodiments, X¹⁴ and the target nucleotide comprise a mismatched base pair. In some embodiments, the mismatched base pair is a wobble base pair. In some embodiments, X¹⁴ is a nucleotide that forms a wobble base pair with the target nucleotide. In some embodiments, X¹⁴ comprises either inosine or uridine. In some embodiments, the wobble base pair is I:C or U:G. In some embodiments, X¹⁴ comprises inosine if the target nucleotide comprises cytidine. In some embodiments, X¹⁴ comprises uridine if the target nucleotide comprises guanosine.

In some embodiments, X⁰¹ and a nucleotide at a corresponding position on the target mRNA comprise a mismatched base pair. In some embodiments, the mismatched base pair is A:G or U:C. In some embodiments, X⁰¹ comprises either adenosine or uridine. In some embodiments, X⁰¹ comprises adenosine if the nucleotide at the corresponding position on the target mRNA comprises guanosine. In some embodiments, X⁰¹ comprises uridine if the nucleotide at the corresponding position on the target mRNA comprises cytidine.

In some embodiments, the antisense strand of Formula (I) comprises at least one modified nucleotide and/or at least one modified internucleotide linkage. In some embodiments, the antisense strand comprises one or more nucleoside modifications selected from 2′-aminoethyl, 2′-fluoro, 2′-O-methyl, 2′-O-methoxyethyl, and 2′-deoxy-2′-fluoro-β-d-arabinonucleic acid. In some embodiments, the antisense strand comprises at least one phosphorothioate internucleotide linkage. In some embodiments, the nucleic acid further comprises at least one targeting moiety (e.g., N-acetylgalactosamine (GalNAc)) conjugated to the antisense strand of Formula (I). In some embodiments, the at least one targeting moiety is conjugated to the antisense strand by a cleavable linker.

In some embodiments, the nucleic acid further comprises a sense strand of 15 to 40 nucleotides in length, where the sense strand forms a duplex region with the antisense strand. In some embodiments, the duplex region comprises a canonical or non-canonical base pairing between a nucleotide on the sense strand and X¹⁴. In some embodiments, the nucleotide on the sense strand comprises cytidine, adenosine, or uridine, if X¹⁴ comprises inosine. In some embodiments, the nucleotide on the sense strand comprises adenosine if X¹⁴ comprises uridine. In some embodiments, the duplex region excludes each instance of N.

In some embodiments, the antisense strand is of Formula (II):

5′-X⁰¹X⁰²X⁰³X⁰⁴X⁰⁵X⁰⁶X⁰⁷X⁰⁸X⁰⁹X¹⁰X¹¹X¹²X¹³X¹⁴X¹⁵X¹⁶X¹⁷X¹⁸X¹⁹X²⁰X²¹-3′ (II),

where: X⁰¹-X¹³ and X¹⁵—X²¹ are each independently any type of nucleotide, with the proviso that X⁰¹-X²¹ is at least 90% complementary to the contiguous nucleotide sequence of the target mRNA; and X¹⁴ is a nucleotide comprising inosine or uridine.

In some embodiments, a nucleic acid of the disclosure is a small interfering RNA (siRNA). In some embodiments, the nucleic acid is a short hairpin RNA (shRNA).

In some aspects, the disclosure provides a composition comprising a nucleic acid described herein and a counterion. In some aspects, the disclosure provides a composition comprising a nucleic acid described herein and a pharmaceutically acceptable carrier.

In some aspects, the disclosure provides a method of reducing expression of a target mRNA in a cell. In some embodiments, the method comprises contacting the cell with a nucleic acid or a composition of the disclosure. In some embodiments, the cell is a mammalian cell. In some embodiments, the mammalian cell is a human cell or a non-human primate cell. In some embodiments, the cell is contacted with the nucleic acid or the composition in vivo. In some embodiments, the cell is contacted with the nucleic acid or the composition in vitro. In some embodiments, the target mRNA encodes a mutant protein. In some embodiments, the mutant protein comprises one or more mutations relative to a wild-type variant. In some embodiments, the target mRNA encodes a protein that is overexpressed in the cell. In some embodiments, the protein is overexpressed relative to a reference expression level (e.g., relative to a wild-type variant, relative to a normal healthy cell). In some embodiments, the target mRNA is a transcript of a gene selected from Angiotensinogen (AGT), Proprotein Convertase Subtilisin/Kexin Type (9PCSK9), Compliment Factor B, Diacylglycerol O-Acyltransferase 2 (DGAT2), and Microtubule Associated Protein Tau (MAPT). In some embodiments, the gene encodes a mutant protein relative to a corresponding wild-type sequence. In some embodiments, the gene encodes a wild-type protein.

In some aspects, the disclosure provides a method of treating a subject. In some embodiments, the method comprises administering to the subject a nucleic acid of the disclosure. In some embodiments, the subject is known to have, or is suspected of having, a disease or condition associated with a target mRNA of the nucleic acid. In some embodiments, the subject is known to have, or is suspected of having, the target mRNA. In some embodiments, the subject is a human. In some embodiments, the subject is a non-human animal (e.g., mouse, rat, rabbit, dog, cat, pig, or non-human primate, such as a monkey or chimpanzee). In some embodiments, the target mRNA encodes a mutant protein. In some embodiments, the mutant protein comprises one or more mutations relative to a wild-type variant. In some embodiments, the target mRNA encodes a protein that is overexpressed in the cell. In some embodiments, the protein is overexpressed relative to a reference expression level (e.g., relative to a wild-type variant, relative to a normal healthy cell). In some embodiments, the subject is known to have, or is suspected of having, a disease or condition associated with a gene selected from Angiotensinogen (AGT), Proprotein Convertase Subtilisin/Kexin Type (9PCSK9), Compliment Factor B, Diacylglycerol O-Acyltransferase 2 (DGAT2), and Microtubule Associated Protein Tau (MAPT). In some embodiments, the target mRNA is a transcript of the gene. In some embodiments, the gene encodes a mutant protein relative to a corresponding wild-type sequence. In some embodiments, the gene encodes a wild-type protein.

The details of certain embodiments of the invention are set forth in the Detailed Description, as described below. Other features, objects, and advantages of the invention will be apparent from the Examples, Drawings, and Claims.

### BRIEF DESCRIPTION OF THE DRAWINGS

The accompanying drawings, which constitute a part of this specification, illustrate several non-limiting embodiments of the invention and together with the description, serve to explain the principles of the invention.

FIG. 1 shows example nucleic acid structures in which antisense strands are in duplex with a sense strand or a target RNA strand.

FIG. 2 shows an example formula for a nucleic acid having a sense strand (shown 5′ to 3′) and an antisense strand (shown 3′ to 5′).

FIGS. 3A-3B show results from in vivo testing of siRNA (RD1354) in cynomolgus monkeys. Results for individual monkeys are shown in FIG. 3A, with averaged results for the group shown in FIG. 3B.

### DETAILED DESCRIPTION

Aspects of the disclosure relate to the discovery that an effective reduction in target RNA levels can be achieved using an antisense strand configured to mediate a non-canonical interaction (e.g., a mismatch interaction, such as a wobble base-pairing) between its position 14 nucleotide and the target RNA. In some aspects, the disclosure provides new strategies for the design of effective antisense molecules, supplementing conventional guidelines to allow for a greater number and variety of potential target mRNA sites without sacrificing efficiency.

The efficiency of short interfering RNA (siRNA) molecules depends on different factors, including target availability, secondary structures of mRNA, position of matching and intrinsic characteristics of siRNA and mRNA. Precise design of siRNAs is a critical step owing to the fact that only a few changes in the nucleotides within the sequence can alter its functionality.

The inventors have recognized and appreciated that conventional siRNA design strategies follow certain rules which can limit the number and variety of potential RNA target sites. In some aspects, the disclosure overcomes certain of these limitations by providing nucleic acids comprising an antisense strand having, at position 14 from its 5′ end, an abasic site or a nucleotide that does not form a canonical base pair with a target nucleotide at a corresponding position on the target RNA. In some embodiments, a non-canonical interaction, such as a wobble base pair, is formed between the position 14 nucleotide and a G or C nucleotide on the target RNA. Thus, in some embodiments, the wobble base pair and other non-canonical interactions of the disclosure provide an alternative design strategy to the conventional preference for A or U nucleotides at this position on the target molecule. In some aspects, the disclosure relates to the surprising discovery that nucleic acids that form such a wobble base pair with a target RNA resulted in a highly effective reduction in target RNA levels.

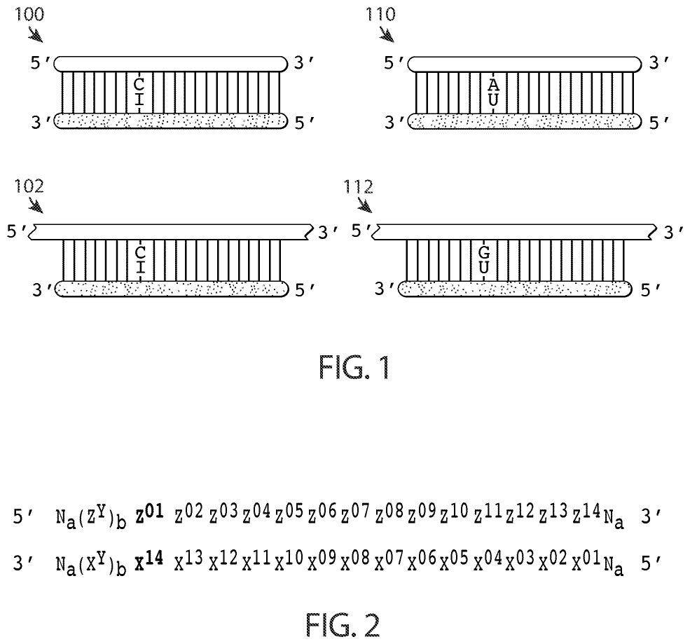

FIG. 1 shows example nucleic acid structures in which antisense strands (stippled shapes) are in duplex with a sense or target strand (solid shapes). In some embodiments, a nucleic acid comprising an antisense strand in duplex with a sense strand may generally be referred to herein as an RNA silencing agent. Examples of RNA silencing agents are provided elsewhere herein and include, without limitation, siRNA and shRNA.

RNA silencing agent 100 is shown having an antisense strand (stippled shape) in duplex with a sense strand (solid shape). As used herein, in some embodiments, an antisense strand of an RNA silencing agent refers to a strand having a region of complementarity to a target strand (e.g., a target RNA, such as mRNA). In some embodiments, the region of complementarity has a nucleotide sequence sufficiently complementary to the desired target strand to direct target-specific silencing, e.g., complementarity sufficient to trigger the destruction of the desired target strand by the RNAi machinery or process (RNAi interference) or complementarity sufficient to trigger translational repression of the desired target mRNA.

As shown, RNA silencing agent 100 comprises inosine at the position 14 nucleotide on the antisense strand. As used herein, in some embodiments, the position 14 nucleotide refers to a nucleotide on an antisense strand that is capable of forming a non-canonical interaction, such as a wobble base pair, with a G or C nucleotide at a corresponding position on a target strand. In some embodiments, the position 14 nucleotide on an antisense strand is numbered relative to its 5′ end, where the 5′-most nucleotide on the antisense strand can be designated as the position 1 nucleotide. In some embodiments, the position 14 nucleotide on the antisense strand is numbered relative to its region of complementarity to a target strand, where the 5′-most nucleotide of the region of complementarity can be designated as the position 1 nucleotide. For example, in some embodiments, an RNA silencing agent comprises an antisense strand having one or more nucleotides in a 5′ overhang region relative to the sense strand. In this context, the position 14 nucleotide is numbered relative to the 5′-most nucleotide that is not in the overhang region, the latter of which can be designated as the position 1 nucleotide.

As generally depicted, the inosine at the position 14 nucleotide on the antisense strand of RNA silencing agent 100 forms a wobble base pair with a cytidine at a corresponding position on the sense strand. While RNA silencing agent 100 shows cytidine at the corresponding position on the sense strand by way of example, other nucleosides can be utilized at this position. For example, since inosine can form a wobble base pair with cytidine, adenosine, or uridine, the nucleotide at the corresponding position on the sense strand can comprise any one of these nucleosides. However, as described herein, the position 14 nucleotide can advantageously form a non-canonical interaction (e.g., a wobble base pair) with a corresponding position on a target RNA. Thus, it should be appreciated that, in the context of an RNA silencing agent 100, the position 14 nucleotide on the antisense strand need not form a wobble base pair with the nucleotide at the corresponding position on the sense strand. For example, in some embodiments, the nucleotide at the corresponding position on the sense strand of the RNA silencing agent comprises a nucleoside that does not base pair with the inosine of the position 14 nucleotide. Accordingly, in some embodiments, the corresponding position on the sense strand of RNA silencing agent 100 can comprise any nucleoside (e.g., adenosine, guanosine, cytidine, uridine, thymidine, inosine, or an analog thereof) which may or may not base pair with the inosine of the position 14 nucleotide.

Target duplex 102 shows the antisense strand (stippled shape) of RNA silencing agent 100 in duplex with a target strand (solid shape). In some embodiments, the target strand is a target RNA (e.g., mRNA). As generally depicted, the inosine of the position 14 nucleotide on the antisense strand forms a wobble base pair with a cytidine at a corresponding position on the target strand. In accordance with the disclosure, the wobble base pair of I:C provides an advantageous alternative to the otherwise unfavorable G:C base pair at this position. As described herein, in some embodiments, the antisense strand comprises a region of complementarity, which refers to the nucleotides of the antisense strand that form base pairs with nucleotides of the target strand.

In some embodiments, position 14 on the antisense strand can comprise an abasic site or a nucleotide that does not form a canonical base pair with a target nucleotide at a corresponding position on the target strand. Target duplex 102 depicts an example in which inosine at position 14 on the sense strand forms a wobble base pair with cytidine at the corresponding position on the target strand. It should be appreciated that, in some embodiments, a wobble base pair is one example of a mismatched base pair in accordance with the disclosure. Accordingly, in some embodiments, where the target nucleotide comprises cytidine, the position 14 nucleotide comprises a nucleoside other than guanosine. For example, in some embodiments, the target nucleotide comprises cytidine, and the position 14 nucleotide comprises adenosine, uridine, or cytidine. In some embodiments, however, position 14 on the antisense strand comprises an abasic site such that a nucleobase is absent at this position.

RNA silencing agent 110 is shown having an antisense strand (stippled shape) in duplex with a sense strand (solid shape), where the position 14 nucleotide comprises uridine. In this example, the sense strand of RNA silencing agent 110 comprises adenosine at a position corresponding to the uridine of the position 14 nucleotide. As described with respect to RNA silencing agent 100, nucleotide complementarity at this position is not a requirement for RNA silencing agent 110, as the advantages described herein relate to the non-canonical interaction (e.g., a wobble base pair) formed at this position in the context of a target duplex. Accordingly, in some embodiments, the corresponding position on the sense strand of RNA silencing agent 110 can comprise any nucleoside (e.g., adenosine, guanosine, cytidine, uridine, thymidine, inosine, or an analog thereof) which may or may not base pair with the uridine of the position 14 nucleotide.

Target duplex 112 shows the antisense strand (stippled shape) of RNA silencing agent 110 in duplex with a target strand (solid shape). In some embodiments, the target strand is a target RNA (e.g., mRNA). As generally depicted, the uridine of the position 14 nucleotide on the antisense strand forms a wobble base pair with a guanosine at a corresponding position on the target strand. In accordance with the disclosure, the wobble base pair of U:G provides an advantageous alternative to the otherwise unfavorable C:G base pair at this position.

Target duplex 112 depicts an example in which uridine at position 14 on the sense strand forms a wobble base pair with guanosine at the corresponding position on the target strand. It should be appreciated that, in some embodiments, a wobble base pair is one example of a mismatched base pair in accordance with the disclosure. Accordingly, in some embodiments, where the target nucleotide comprises guanosine, the position 14 nucleotide comprises a nucleoside other than cytidine. For example, in some embodiments, the target nucleotide comprises guanosine, and the position 14 nucleotide comprises adenosine, guanosine, or uridine. In some embodiments, however, position 14 on the antisense strand comprises an abasic site such that a nucleobase is absent at this position.

The nucleic acid structures of FIG. 1 are generically depicted and should not be construed as limiting to the disclosure. For example, RNA silencing agents 100 and 110 are each shown as having an antisense strand of 21 nucleotides in length which is fully complementary to a sense strand. Similarly, target duplexes 102 and 112 are each shown as having an antisense strand of 21 nucleotides in length which is fully complementary to a target strand. It should be appreciated that these examples are provided for illustrative purposes, and an antisense or sense strand may be of more or fewer than 21 nucleotides in length, and the degree of complementarity of an RNA silencing agent or target duplex may be less than 100%, as described elsewhere herein.

FIG. 2 shows an example formula for an RNA silencing agent having a sense strand (shown 5′ to 3′) and an antisense strand (shown 3′ to 5′). The variables N, X, and Z denote individual nucleotides, and the variables a and b are defined herein. In some embodiments, the RNA silencing agent comprises a duplex region formed by base pair interactions between the sense strand at (Z^{Y})_{b}—Z¹⁴ and the antisense strand at (X^{Y})_{b}—X⁰¹. In some embodiments, b is an integer from 1-17, inclusive. By way of example, for a duplex region of 21 nucleotides in length, such as that shown for RNA silencing agents 100 and 110, b is 7.

In some embodiments, (X^{Y})_{b} and X¹³—X⁰¹ are each independently any type of nucleotide, with the proviso that (X^{Y})_{b}—X⁰¹ is at least 80% complementary to (Z^{Y})_{b}—Z¹⁴. Accordingly, in some embodiments, the duplex region of an RNA silencing agent refers to sequences of the sense and antisense strands that are at least 80% complementary (e.g., at least 85%, at least 90%, at least 95%, or 100% complementary).

In some embodiments, an RNA silencing agent comprises at least one overhang region as denoted by Nₐ in FIG. 2. In some embodiments, a is independently an integer from 0-2, such that the RNA silencing agent can optionally comprise at least one overhang of up to 2 nucleotides. As used herein, in some embodiments, an overhang refers to terminal non-base pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of an RNA silencing agent. In some embodiments, the overhang is a 5′ or 3′ overhang on the antisense strand or sense strand of an RNA silencing agent. In some embodiments, an RNA silencing agent comprises a 5′ overhang and a 3′ overhang on the sense strand. In some embodiments, an RNA silencing agent comprises a 3′ overhang on the sense strand and a 3′ overhang on the antisense strand. In some embodiments, an RNA silencing agent comprises a 3′ overhang on the sense strand, a 3′ overhang on the antisense strand, and neither a 5′ overhang on the sense strand nor a 5′ overhang on the antisense strand. Although not depicted, it should be appreciated that, in some embodiments, an RNA silencing agent having a 3′ overhang on the antisense strand may be configured such that the 3′ overhang is removable (e.g., cleavable) from the RNA silencing agent.

In some embodiments, an RNA silencing agent comprises at least one stem-loop. In some embodiments, a is independently an integer from 0-30, such that the RNA silencing agent can optionally comprise at least one stem-loop of up to 30 nucleotides. Accordingly, in some embodiments, “N” nucleotides denote optional nucleotides forming a stem-loop at either or both ends of the nucleic acid. For example, in some embodiments, the N nucleotides at the 5′ end of one strand and the N nucleotides at the 3′ end of the other strand are covalently connected through a stem-loop having a stem region and a loop region. In some embodiments, a stem region comprises a duplex of between about 1 and up to about 26 base pairs in length. In some embodiments, a loop region comprises a single-stranded portion of between about 4 and up to 10 nucleotides in length.

In some embodiments, an RNA silencing agent comprises an abasic site or a nucleotide, denoted by X¹⁴ in FIG. 2, that does not form a canonical base pair with a target nucleotide at a corresponding position on a target strand (e.g., a target RNA, such as mRNA). As shown, Z⁰¹ is a nucleotide on the sense strand at a position corresponding to X¹⁴ on the antisense strand. In some embodiments, X¹⁴ is an abasic site. In some embodiments, X¹⁴ is adenosine, inosine, or uridine. In some embodiments, Z⁰¹ is any type of nucleotide. In some embodiments, Z⁰¹ is guanosine, cytidine, adenosine, or uridine. In some embodiments, X¹⁴ is inosine, and Z⁰¹ is cytidine, adenosine, or uridine. In some embodiments, X¹⁴ is uridine, and Z⁰¹ is adenosine or guanosine.

In some embodiments, the antisense strand of the RNA silencing agent in FIG. 2 is an antisense strand of Formula (I), as described elsewhere herein.

As described herein, in some embodiments, an RNA silencing agent refers to a nucleic acid comprising an antisense strand having sufficient complementarity to a target strand (e.g., a target RNA sequence) to mediate an RNA-mediated silencing mechanism (e.g. RNAi). In some embodiments, the nucleic acid is a duplex molecule (or a molecule having duplex-like structure) comprising a sense strand and a complementary antisense strand (or portions thereof). In some embodiments, the antisense strand comprises, at position 14 from its 5′ end, a nucleotide that forms a wobble base pair with a nucleotide at a corresponding position on a target strand. In some embodiments, the position 14 nucleotide comprises a nucleoside selected from inosine and uridine.

In some embodiments, the term nucleoside refers to a molecule having a purine or pyrimidine base covalently linked to a ribose or deoxyribose sugar. A nucleoside consists of a nucleobase (e.g., a nitrogenous base (e.g., nucleobase)) and a pentose sugar (e.g., ribose). The pentose sugar can be either ribose or deoxyribose. Nucleosides are the biochemical precursors of nucleotides, which are the constituent components of RNA and DNA. The term “nucleotide,” as may be used herein, refers to a nucleobase and a pentose sugar (i.e., nucleoside), and one or more phosphate groups. In a nucleoside, the anomeric carbon is linked through a glycosidic bond to the N9 of a purine or the N1 of a pyrimidine. Examples of nucleosides and nucleobases include, without limitation, cytidine (C), uridine (U), adenosine (A), guanosine (G), thymidine (T), and inosine (I), however it is also to be understood that the term describes nucleosides which result from modification (as such term is defined herein) as they contain a nucleobase and a pentose sugar. For example, nucleosides include, natural nucleosides (e.g., deoxyadenosine, deoxythymidine, deoxyguanosine, and deoxycytidine), nucleoside analogs (e.g., 2-aminoadenosine, 2-thiothymidine, inosine, pyrrolo pyrimidine, 3-methyl adenosine, 5-methylcytidine, C5 bromouridine, C5-fluorouridine, C5-iodouridine, C5-propynyl uridine, C5-propynyl cytidine, C5-methylcytidine, 7-deazaadenosine, 7-deazaguanosine, 8-oxoadenosine, 8-oxoguanosine, 0(6)-methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′ fluororibose, ribose, 2′ deoxyribose, 2′ O methylcytidine, arabinose, and hexose), or modified phosphate groups (e.g., phosphorothioates and 5′ N phosphoramidite linkages), xanthine, hypoxanthine, nubularine, isoguanisine, tubercidine, 2-aminopurine, 2,6-diaminopurine, 3-deazaadenosine, 7-deazaadenosine, 7-methyladenosine, 8-azidoadenosine, 8-methyladenosine, 5-hydroxymethylcytosine, 5-methylcytidine, Pyrrolocytidine, 7-aminomethyl-7-deazaguanosine, 7-deazaguanosine, 7-methylguanosine, 8-aza-7-deazaguanosine, thienoguanosine, inosine, 4-thio-uridine, 5-methoxyuridine, dihydrouridine, and pseudouridine. In some embodiments, the term nucleotide refers to a nucleoside having one or more phosphate groups joined in ester linkages to the sugar moiety. Examples of nucleotides include nucleoside monophosphates, diphosphates, and triphosphates. In some embodiments, the term nucleic acid refers to a polymer of nucleotides joined together by a phosphodiester or phosphorothioate linkage between 5′ and 3′ carbon atoms. As used herein, in some embodiments, a nucleic acid can refer to a single-stranded molecule, or a nucleic acid can refer to a double-stranded molecule (e.g., a sense strand in duplex with an antisense strand).

In some embodiments, a nucleic acid of the disclosure comprises an antisense strand of at least 19 nucleotides in length. For example, in some embodiments, an antisense strand is 19 to 31 nucleotides in length (e.g., 19 to 25, 19 to 21, 21 to 31, 21 to 25, 19, 20, 21, 22, 23, 24, or 25, nucleotides in length). In some embodiments, an antisense strand comprises a region of complementarity to a target strand (e.g., a target mRNA). In some embodiments, the region of complementarity refers to a nucleotide sequence of the antisense strand that is at least 80% (e.g., at least 85%, at least 90%, at least 95%, or 100%) complementary to a contiguous sequence of a target mRNA. In some embodiments, the region of complementarity is 19 to 31 nucleotides in length (e.g., 19 to 25, 19 to 21, 21 to 31, 21 to 25, 19, 20, 21, 22, 23, 24, or 25, nucleotides in length).

In some embodiments, a nucleic acid of the disclosure comprises a sense strand that forms a duplex region with an antisense strand. In some embodiments, a sense strand is at least 19 nucleotides in length. For example, in some embodiments, a sense strand is 19 to 40 nucleotides in length (e.g., 19 to 35, 19 to 30, 19 to 25, 19 to 21, 21 to 30, 25 to 30, or 30 to 40, nucleotides in length). In some embodiments, a duplex region refers to a structure formed through complementary base-pairing of two antiparallel sequences of nucleotides. In some embodiments, a duplex region formed between sense and antisense strands is at least 80% (e.g., at least 85%, at least 90%, at least 95%, or 100%) complementary.

In some embodiments a duplex region comprises at least one mismatched base pair of the duplex (e.g., nucleotides which do not base pair according to conventional Watson-Crick base pairing rules). In some embodiments, a mismatch of the at least one mismatched base pair comprises the position 14 nucleotide on an antisense strand, as described herein. For example, in some embodiments, the position 14 nucleotide on an antisense strand may form a mismatched base pair with a corresponding nucleotide on an antisense strand (e.g., in a duplex region) and/or a target nucleotide at a corresponding position on a target strand. In some embodiments, a duplex region contains more than one mismatch. In some embodiments, a duplex region contains fewer than 30 mismatches. In some embodiments, a duplex region contains more than one mismatch, but fewer than 30 mismatches. In some embodiments, a duplex region contains at least one, but fewer than 11 mismatches. In some embodiments, a duplex region contains at least one, but fewer than 6 mismatches. In some embodiments, a duplex region contains at least one, but fewer than 4 mismatches. In some embodiments, where a duplex region contains more than one mismatch, the mismatches are consecutive (e.g., adjacent) in the nucleic acid. In some embodiments, where a duplex region contains more than one mismatch, the mismatches are non-consecutive (e.g., not adjacent) in the nucleic acid. In some embodiments, where a duplex region contains more than two mismatches, there is at least one grouping of two or more mismatches adjacent to one another. In some embodiments, where a duplex region contains more than two mismatches, there are no two or more mismatches adjacent to one another. In some embodiments, the duplex region does not comprise a mismatch. In some embodiments, a mismatch of the duplex region comprises a wobble base pair.

In some embodiments, a duplex region comprises one or more wobble base pairs. In some embodiments, a wobble base pair of the one or more wobble base pair comprises the position 14 nucleotide on an antisense strand, as described herein. For example, in some embodiments, the position 14 nucleotide on an antisense strand may form a wobble base pair with a corresponding nucleotide on an antisense strand (e.g., in a duplex region) and/or a target nucleotide at a corresponding position on a target strand. In some embodiments, a wobble base pair is a term of art generally known to refer to a base pairing of specific nucleotides (e.g., a wobble base pair), which are non-canonical in that they are not Watson-Crick base pairs (e.g., are a form of, or subset of, mismatched base pairs). Specifically, the term wobble is used as a term to describe base pairings of hypoxanthine (inosine (I)) and uracil (U) (I:U base pair); guanine (G) and U (G:U base pair); I and adenine (A) (I:A base pair); and I and cytosine (C) (I:C base pair).

In some embodiments, a sense strand and/or an antisense strand comprises at least one modified nucleotide. In some embodiments, a modified nucleotide has one or more chemical modification in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc. Examples of modified nucleotides include, but are not limited to, 2-amino-guanosine, 2-amino-adenosine, 2,6-diamino-guanosine, and 2,6-diamino-adenosine. Examples of positions of the nucleotide which may be derivatized include the 5 position, e.g., 5-(2-amino)propyl uridine, 5-bromo uridine, 5-propyne uridine, 5-propenyl uridine, etc.; the 6 position, e.g., 6-(2-amino)propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs known in the art. In some embodiments, an antisense strand comprises one or more nucleoside modifications selected from 2′-aminoethyl, 2′-fluoro, 2′-O-methyl, 2′-O-methoxyethyl, and 2′-deoxy-2′-fluoro-β-d-arabinonucleic acid.

Additional examples of modified nucleotides in accordance with the disclosure include nucleotides having a modified purine or pyrimidine nucleobase. Purine and/or pyrimidine nucleobases may be modified, for example by amination or deamination of the heterocyclic rings. Further, modified sugars, such as 2′-O substitutions to the sugar (e.g., ribose), including without limitation, 2′-O-methoxyethyl sugar, a 2′-fluoro sugar modification (2′-fluoro), a 2′-O-methyl sugar (2′-O-methyl), 2′-O-ethyl sugar, 2′-Cl, 2′-SH, and substitutions thereof (e.g., 2′-SCH₃), a bicyclic sugar moiety, or substitutions such as a 2′-O moiety with a lower alkyl or substitutions thereof (e.g., —CH₃, —CF₃), 2′-amino or substitutions thereof, 2′,3′-seco nucleotide mimic, 2′-F-arabino nucleotide, inverted nucleotides, inverted 2′-O-methyl nucleotide, 2′-O-deoxy nucleotide, an alkenyl, an alkynyl, a methoxyethyl (2′-O-MOE), an —H (as in DNA), or other substituent, may be introduced. Ribose mimics are also contemplated, such as, without limitation, morpholino, glycol nucleic acid (GNA), UNA, cyclohexenyl nucleic acid (CeNA).

Other examples include, 2′-4′ sugar bridged variants, such as locked-nucleic acids (LNAs), and 2′-O, 4′-C-ethylene-bridged nucleic acid (ENA). Locked nucleic acids are modified RNA nucleotides in which the ribose sugar is modified by means of a bridge connecting the 2′ oxygen and 4′ carbon (often seen as a methylene bridge between the 2′ oxygen and 4′ carbon). This bridge operably “locks” the ribose in the 3′-endo conformation. The locked ribose sugar conformation can enhance base stacking and backbone pre-organization, which can affect (e.g., increase) its hybridization properties (e.g., thermal stability and hybridization specificity). Locked nucleic acids can be inserted into both RNA and DNA oligonucleotides to hybridize with DNA or RNA according to typical Watson-Crick base-pairing rules (i.e., complementarity).

Other chemistries and modification are known in the field of oligonucleotides that can be readily used in accordance with the disclosure and are encompassed within the definition of a nucleic acid modification, for example, the term modification shall further include any alteration, change, or manipulation, which results in the formation of any nucleoside other than the natural nucleosides.

In some embodiments, a nucleic acid comprises more than one nucleoside modification. In some embodiments, a nucleic acid comprises more than two nucleoside modifications. In some embodiments, more than 25%, but less than or equal to 100%, of the nucleosides in a nucleic acid comprise a nucleoside modification. In some embodiments, more than 50% of the nucleosides in a nucleic acid comprise a nucleoside modification. In some embodiments, more than 75%, but less than or equal to 100%, of the nucleosides in a nucleic acid comprise a nucleoside modification. In some embodiments, at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) of the nucleosides in a nucleic acid comprise a nucleoside modification. In some embodiments, at least 95%, but less than or equal to 100%, of the nucleosides in a nucleic acid comprise a nucleoside modification.

In some embodiments, a sense strand and/or an antisense strand comprises at least one modified internucleotide linkage. As used herein, in some embodiments, a modified internucleotide linkage refers to an internucleotide linkage having one or more chemical modifications compared with a reference internucleotide linkage comprising a phosphodiester bond. In some embodiments, a modified internucleotide linkage is a non-naturally occurring linkage. Typically, a modified internucleotide linkage confers one or more desirable properties to a nucleic acid in which the modified internucleotide linkage is present. For example, a modified nucleotide may improve thermal stability, resistance to degradation, nuclease resistance, solubility, bioavailability, bioactivity, reduced immunogenicity, etc. In some embodiments, an antisense strand comprises at least one phosphorothioate internucleotide linkage. Further modification to the linkages include amidation and peptide linkers. Other examples include, phosphodiester, phosphotriester, phosphoro(di)thioate, methylphosphonate, phosphor-amidate linkers, phosphonates, 3′-methylenephosphonate, 5′-methylenephosphonate, Boranophosphate and the like. Further, the chirality of the isomers may be modified (e.g., Rp and Sp).

In some embodiments, a nucleic acid comprises more than two modified internucleotide linkages. In some embodiments, a nucleic acid comprises more than three modified internucleotide linkages. In some embodiments, more than 25% of the internucleotide linkages of a nucleic acid comprise a modification. In some embodiments, more than 50% of the internucleotide linkages of a nucleic acid comprise a modification. In some embodiments, more than 75% of the internucleotide linkages of a nucleic acid comprise a modification. In some embodiments, at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) of the internucleotide linkages in a nucleic acid comprise a modification. In some embodiments, at least 95% of the internucleotide linkages of a nucleic acid comprise a modification.

In some embodiments, a sense strand and/or an antisense strand is conjugated to at least one N-acetylgalactosamine (GalNAc) moiety.

In some embodiments, the disclosure provides a nucleic acid for reducing expression of a target mRNA. In some embodiments, reducing expression of a target mRNA can be achieved by directing target-specific silencing, e.g., by triggering the destruction of the target mRNA by the RNAi machinery or process (RNAi interference), and/or by triggering translational repression of the desired target mRNA.

### EXAMPLES

### Example 1. AGT Gene Expression Assay for siRNA Knockdown

The day before transfections, HepG2 cells were seeded in antibiotic-free media at 10,000 cells/well in a 96-well plate. AGT siRNA was diluted to working stocks of 1 mM and 0.10 mM from a stock solution of 10 mM. Mixes were prepared separately as shown below (amounts shown for triplicates), gently mixed, and incubated at room temperature for 5 minutes.

| | Volume (μL) per reaction |
|---|---|
| Component | (for triplicates) |
| 1 uM (or 0.1 uM) AGT siRNA | 4 |
| Opti-MEM | 36 |
| | 40 |

| Component | Volume (μL) per reaction |
|---|---|
| Dharmafect 4 | 1.6 |
| Opti-MEM | 38.4 |
| | 40 |

The mixtures were combined and incubated at room temperature for 20 minutes. During this incubation, the medium in the 96-well plates was replaced with 80 μL of antibiotic-free medium. A volume of 20 μL of the mixture was added to each well, and plates were tapped gently to mix the contents of the wells. Cells were incubated at 37° C. in 5% C02 for 24 hours (for mRNA analysis).

After 24 hours, Lysis Solution was prepared by combining 49.5 μL/reaction of RT Lysis Solution and 0.5 μL/reaction of DNaseI, multiplied by the number of total reactions. The cell culture medium was aspirated and rinsed with 50 μL of cold PBS. A volume of 50 μL of Lysis Solution/well was added and pipetted to mix, followed by a 5 minute incubation at room temperature. A volume of 5 μL of Stop Solution (room temperature) was added and pipetted to mix, followed by a 2 minute incubation at room temperature. A Master Mix was prepared on ice as shown below.

| Component | Volume (μL) per reaction |
|---|---|
| TaqMan 1-Step qRT-PCR Mix | 5 |
| AGT(FAM) TaqMan Gene | 1 |
| Expression Assay | |
| GAPDH(VIC) TaqMan Gene | 1 |
| Expression Assay | |
| Nuclease-free water | 11 |
| | 18 |

On ice, a volume of 18 μL of Master Mix was added to each well of an optical 96-well PCR plate. A volume of 2 μL of lysate (or water for NTC) was added to each well. The plate was sealed with optical adhesive cover, vortexed 5-10 seconds, and briefly spun to remove air bubbles. A reaction was setup to be run in a QuantStudio3 qPCR machine as shown below.

| | | | |
|---|---|---|---|
| 50° C. | 1 | minute | |
| 95° C. | 20 | seconds | |
| 95° C. | 15 | seconds | 40 cycles |
| 60° C. | 1 | minute | |

Plated were loaded into the qPCR machine, and the reactions were run. After completion of the run, results were downloaded, and the Cq values (same as Ct) were used to analyze the data according to the following: (i) Record Ct values for AGT and GAPDH for each sample; (ii) ΔΔCt=AGT Ct value−GAPDH Ct value; (iii) ΔΔCtRQ=ΔΔCt test sample−ΔΔCt non-transfected test sample; (iv) RQ (expression fold change)=2^{-ΔΔCt}; (v) % AGT remaining=2^{-ΔΔCt}×100; (vi) % AGT knockdown=100−% AGT remaining.

The sequence information for siRNAs evaluated in these experiments is provided below in Table 1.

TABLE 1
siRNA Sequence Information

| | | SEQ |
|---|---|---|
| Identifier^{†} | Sequence^{‡} | ID NO |
| RD1292/ | rU.rC.rC.rA.rC.rC.rU.rC.rA.rU.rC.rA.rU.rC.rC.rA.rC.rA.rA.rU.rG.rA.rU | 1 |
| IS0333 | | |
| RD1292/ | rU.rC.rA.rU.rU.rG.rU.rG.rG.rA.rU.rG.rA.rU.rG.rA.rG.rG.rU.rG.rG | 2 |
| IA0334 | | |
| RD1354/ | H2.mC*mC.mA.fC.mC.mU.fC.mA.mU.fC.fA.fU.mC.mC.fA.mC.mA.fA.mU. | 3 |
| IS0340 | mG*mA*mU | |
| RD1354/ | mU*fC*mA.mU.mU.fG.mU.fG.mG.mA.mU.mG.mA.fU.mG.fA.mG.mG.mU* | 4 |
| IA0336 | fG*mG.mA | |
| RD1276/ | rU.rG.rG.rU.rG.rG.rA.rG.rA.rG.rU.rC.rU.rC.rA.rC.rU.rU.rU.rC.rC.rA.rU | 5 |
| IS0317 | | |
| RD1276/ | rU.rG.rG.rA.rA.rA.rG.rU.rG.rA.rG.rA.rC.rU.rC.rU.rC.rC.rA.rC.rC | 6 |
| IA0318 | | |
| RD1324/ | H2.mG*mG.mU.fG.mG.mA.fG.mA.mG.fU.fC.fU.mC.mA.fC.mU.mU.fU.mC. | 7 |
| IS0336 | mC*mA*mU | |
| RD1324/ | mU*fG*mG.mA.mA.fA.mG.fU.mG.mA.mG.mA.mC.fU.mC.fU.mC.mC.mA* | 8 |
| IA0335 | fC*mC.mA | |
| RD1270/ | rU.rA.rG.rG.rU.rG.rA.rC.rC.rG.rG.rG.rU.rG.rU.rA.rC.rA.rU.rA.rC.rA.rU | 9 |
| IS0311 | | |
| RD1270/ | rU.rG.rU.rA.rU.rG.rU.rA.rC.rA.rC.rC.rC.rI.rG.rU.rC.rA.rC.rC.rU | 10 |
| IA0312 | | |
| RD1271/ | rU.rG.rA.rG.rA.rC.rA.rU.rC.rC.rC.rC.rU.rG.rU.rG.rG.rA.rU.rG.rA.rA.rU | 11 |
| IS0312 | | |
| RD1271/ | rU.rU.rC.rA.rU.rC.rC.rA.rC.rA.rG.rG.rG.rI.rA.rU.rG.rU.rC.rU.rC | 12 |
| IA0313 | | |
| RD1272/ | rU.rA.rC.rC.rC.rU.rG.rG.rC.rC.rU.rC.rU.rC.rU.rC.rU.rA.rU.rC.rU.rA.rU | 13 |
| IS0313 | | |
| RD1272/ | rU.rA.rG.rA.rU.rA.rG.rA.rG.rA.rG.rA.rG.rI.rC.rC.rA.rG.rG.rG.rU | 14 |
| IA0314 | | |
| RD1273/ | rU.rC.rA.rC.rC.rC.rU.rG.rA.rC.rU.rU.rU.rC.rA.rA.rC.rA.rC.rC.rU.rA.rU | 15 |
| IS0314 | | |
| RD1273/ | rU.rA.rG.rG.rU.rG.rU.rU.rG.rA.rA.rA.rG.rU.rC.rA.rG.rG.rG.rU.rG | 16 |
| IA0315 | | |
| RD1274/ | rU.rC.rC.rU.rU.rC.rC.rA.rA.rC.rA.rC.rU.rG.rG.rA.rG.rU.rG.rA.rC.rA.rU | 17 |
| IS0315 | | |
| RD1274/ | rU.rG.rU.rC.rA.rC.rU.rC.rC.rA.rG.rU.rG.rU.rU.rG.rG.rA.rA.rG.rG | 18 |
| IA0316 | | |
| RD1275/ | rU.rC.rU.rC.rA.rA.rG.rU.rA.rC.rC.rC.rU.rU.rC.rA.rC.rU.rG.rA.rG.rA.rU | 19 |
| IS0316 | | |
| RD1275/ | rU.rC.rU.rC.rA.rG.rU.rG.rA.rA.rG.rG.rG.rU.rA.rC.rU.rU.rG.rA.rG | 20 |
| IA0317 | | |
| RD1276/ | rU.rG.rG.rU.rG.rG.rA.rG.rA.rG.rU.rC.rU.rC.rA.rC.rU.rU.rU.rC.rC.rA.rU | 21 |
| IS0317 | | |
| RD1276/ | rU.rG.rG.rA.rA.rA.rG.rU.rG.rA.rG.rA.rC.rU.rC.rU.rC.rC.rA.rC.rC | 22 |
| IA0318 | | |
| RD1278/ | rU.rG.rA.rA.rC.rC.rG.rC.rC.rC.rA.rU.rU.rC.rC.rU.rG.rU.rU.rU.rG.rA.rU | 23 |
| IS0319 | | |
| RD1278/ | rU.rC.rA.rA.rA.rC.rA.rG.rG.rA.rA.rU.rG.rI.rG.rC.rG.rG.rU.rU.rC | 24 |
| IA0320 | | |
| RD1279/ | rU.rG.rC.rC.rC.rA.rU.rU.rC.rC.rU.rG.rU.rU.rU.rG.rC.rU.rG.rU.rG.rU.rU | 25 |
| IS0320 | | |
| RD1279/ | rA.rC.rA.rC.rA.rG.rC.rA.rA.rA.rC.rA.rG.rI.rA.rA.rU.rG.rG.rG.rC | 26 |
| IA0321 | | |
| RD1280/ | rU.rC.rC.rU.rG.rU.rU.rU.rA.rC.rU.rG.rU.rG.rU.rA.rU.rG.rA.rU.rC.rA.rU | 27 |
| IS0321 | | |
| RD1280/ | rU.rG.rA.rU.rC.rA.rU.rA.rC.rA.rC.rA.rG.rU.rA.rA.rA.rC.rA.rG.rG | 28 |
| IA0322 | | |
| RD1281/ | rU.rA.rA.rG.rC.rA.rG.rC.rC.rG.rU.rU.rU.rC.rU.rC.rC.rU.rU.rG.rG.rU.rU | 29 |
| IS0322 | | |
| RD1281/ | rA.rC.rC.rA.rA.rG.rG.rA.rG.rA.rA.rA.rC.rI.rG.rC.rU.rG.rC.rU.rU | 30 |
| IA0323 | | |
| RD1282/ | rU.rG.rC.rU.rG.rC.rA.rU.rA.rG.rA.rG.rU.rG.rA.rG.rC.rA.rG.rU.rA.rG.rU | 31 |
| IS0323 | | |
| RD1282/ | rC.rU.rA.rC.rU.rG.rC.rU.rC.rA.rC.rU.rC.rU.rA.rU.rG.rC.rA.rG.rC | 32 |
| IA0324 | | |
| RD1283/ | rU.rU.rA.rG.rC.rG.rC.rG.rA.rG.rA.rC.rU.rA.rC.rU.rG.rU.rU.rC.rC.rA.rU | 33 |
| IS0324 | | |
| RD1283/ | rU.rG.rG.rA.rA.rC.rA.rG.rU.rA.rG.rU.rC.rU.rC.rG.rC.rG.rC.rU.rA | 34 |
| IA0325 | | |
| RD1284/ | rU.rC.rA.rG.rU.rG.rU.rU.rC.rC.rC.rU.rU.rU.rU.rC.rA.rA.rG.rU.rU.rG.rU | 35 |
| IS0325 | | |
| RD1284/ | rC.rA.rA.rC.rU.rU.rG.rA.rA.rA.rA.rG.rG.rI.rA.rA.rC.rA.rC.rU.rG | 36 |
| IA0326 | | |
| RD1285/ | rU.rA.rG.rU.rG.rU.rU.rC.rC.rC.rU.rU.rU.rU.rC.rA.rA.rG.rU.rU.rG.rA.rU | 37 |
| IS0326 | | |
| RD1285/ | rU.rC.rA.rA.rC.rU.rU.rG.rA.rA.rA.rA.rG.rI.rG.rA.rA.rC.rA.rC.rU | 38 |
| IA0327 | | |
| RD1286/ | rU.rC.rG.rU.rG.rU.rU.rC.rC.rC.rU.rU.rU.rU.rC.rA.rA.rG.rU.rU.rG.rA.rU | 39 |
| IS0327 | | |
| RD1286/ | rU.rC.A.rA.rC.rU.rU.rG.rA.rA.rA.rA.rG.rI.rG.rA.rA.rC.rA.rC.rG | 40 |
| IA0328 | | |
| RD1287/ | rU.rU.rU.rG.rC.rA.rU.rU.rA.rC.rC.rU.rU.rC.rG.rG.rU.rU.rU.rG.rU.rA.rU | 41 |
| IS0328 | | |
| RD1287/ | rU.rA.rC.rA.rA.rA.rC.rC.rG.rA.rA.rG.rG.rU.rA.rA.rU.rG.rC.rA.rA | 42 |
| IA0329 | | |
| RD1288/ | rU.rC.rU.rG.rC.rA.rU.rU.rA.rC.rC.rU.rU.rC.rG.rG.rU.rU.rU.rG.rU.rA.rU | 43 |
| IS0329 | | |
| RD1288/ | rU.rA.rC.rA.rA.rA.rC.rC.rG.rA.rA.rG.rG.rU.rA.rA.rU.rG.rC.rA.rG | 44 |
| IA0330 | | |
| RD1289/ | rU.rC.rG.rC.rC.rU.rU.rC.rA.rG.rU.rU.rU.rG.rU.rA.rU.rU.rU.rA.rG.rU.rU | 45 |
| IS0330 | | |
| RD1289/ | rA.rC.rU.rA.rA.rA.rU.rA.rC.rA.rA.rA.rC.rU.rG.rA.rA.rG.rG.rC.rG | 46 |
| IA0331 | | |
| RD1290/ | rU.rU.rG.rA.rC.rC.rU.rC.rC.rG.rU.rG.rU.rA.rG.rU.rG.rU.rC.rU.rG.rU.rU | 47 |
| IS0331 | | |
| RD1290/ | rA.rC.rA.rG.rA.rC.rA.rC.rU.rA.rC.rA.rC.rI.rG.rA.rG.rG.rU.rC.rA | 48 |
| IA0332 | | |
| RD1291/ | rU.rC.rG.rA.rC.rC.rU.rC.rC.rG.rU.rG.rU.rA.rG.rU.rG.rU.rC.rU.rG.rU.rU | 49 |
| IS0332 | | |
| RD1291/ | rA.rC.rA.rG.rA.rC.rA.rC.rU.rA.rC.rA.rC.rI.rG.rA.rG.rG.rU.rC.rG | 50 |
| IA0333 | | |

^{†}Each “RD” identifier includes sequence information for a sense strand (“IS” sub-identifier) and
an antisense strand (“IA” sub-identifier).
^{‡}Sequence notation is as follows:
unmodified RNA: rA, rU, rC, rG; 2'-O-methyl RNA: mA, mG, mC, mU; 2′-fluoro RNA: fA, fU, fG, fC;
H2: GalNAc moiety;
“.” denotes a phosphate (phosphodiester) linkage; “*” denotes a phosphorothioate linkage.

The in vitro results from AGT knockdown experiments with siRNA molecules are shown below, in Table 2.

TABLE 2
In vitro siRNA Results

| | Average % AGT |
|---|---|
| siRNA | Knockdown (10 nM) |
| RD1270 | 66 |
| RD1271 | 39 |
| RD1272 | 59 |
| RD1273 | 83 |
| RD1274 | 69 |
| RD1275 | 71 |
| RD1276 | 73 |
| RD1278 | 81 |
| RD1279 | 62 |
| RD1280 | 75 |
| RD1281 | 86 |
| RD1282 | 40 |
| RD1283 | 84 |
| RD1284 | 78 |
| RD1285 | 87 |
| RD1286 | 87 |
| RD1287 | 36 |
| RD1288 | 39 |
| RD1289 | 22 |
| RD1290 | 21 |
| RD1291 | 27 |
| RD1292 | 70 |
| RD1324 | 65 |
| RD1354 | 44 |

A list of materials used in this example are as follows: HepG2 cells (ATCC Cat #HB-8065); AGT siRNA SMARTpool (Dharmacon Cat #L-010988-00-0005); Dharmafect 4 (Dharmacon Cat #T-2004-01); Cells-To-CT 1 Step TaqMan Kit (Fisher Cat #A25603); AGT TaqMan Gene Expression Assay 250 rxns—Hs01586213_m1 (Fisher Cat #4331182); GAPDH TaqMan Gene Expression Assay 250 rxns—Hs02786624_g1 (Fisher Cat #4331182); Nuclease-free water; MicroAmp Optical 96-well plate, 0.2 mL (10 plates) (Fisher Cat #N8010560); MicroAmp Optical Adhesive covers (100) (Fisher Cat #4311971).

### Example 2. In Vivo Testing of RD1354 siRNA

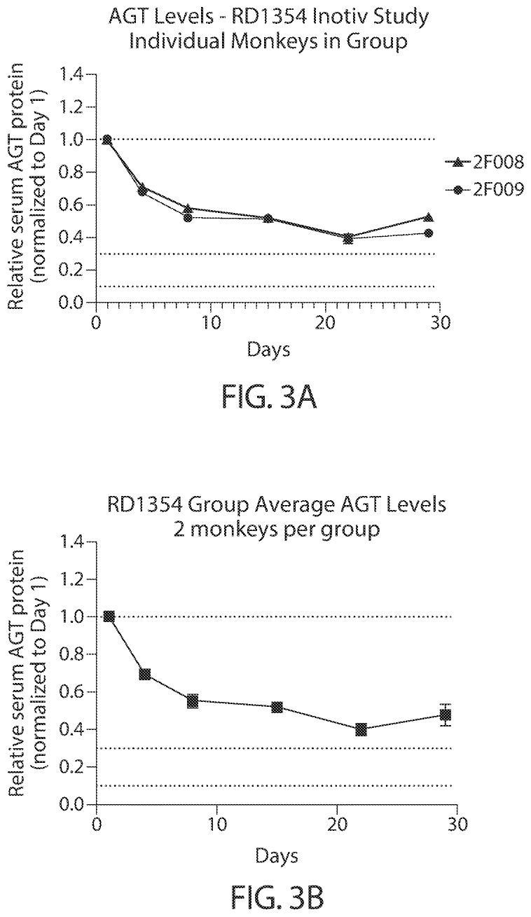

The siRNA “RD1354” was evaluated in cynomolgus monkeys. Prior to the study, the monkeys were kept in quarantine, during which the animals were observed daily for general health. Two cynomolgus monkeys were injected with a single 3 mg/kg subcutaneous dose of oligonucleotide on Day 1 of the study. During the study period, the monkeys were observed daily for signs of illness or distress. Animals were bled on day −6 and on days 1 (prior to dosing), 4, 8, 15, 22, 29, 36, and 43 for serum analysis. Circulating AGT levels were quantified using an ELISA specific for human angiotensinogen (and cross-reactive with cynomolgus), according to manufacturer's protocol (IBL America #27412). Data were expressed as percent of baseline value (Day 1 prior to dosing) and presented as mean plus/minus standard deviation. Results for individual monkeys are shown in FIG. 3A, with averaged results for the group shown in FIG. 3B.

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B,” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

What is claimed is:

1. A nucleic acid for reducing expression of a target mRNA, the nucleic acid comprising an antisense strand of 15 to 31 nucleotides in length having a sequence that is at least 90% complementary to a contiguous sequence of the target mRNA, wherein the sequence of the antisense strand comprises, at position 14 from its 5′ end, an abasic site or a nucleotide that does not form a canonical base pair with a target nucleotide at a corresponding position on the contiguous sequence of the target mRNA.

2. The nucleic acid of claim 1, wherein the target nucleotide comprises either cytidine or guanosine.

3. The nucleic acid of claim 1 or 2, wherein the nucleotide at position 14 on the antisense strand and the target nucleotide comprise a mismatched base pair.

4. The nucleic acid of claim 3, wherein the mismatched base pair is a wobble base pair.

5. The nucleic acid of claim 1 or 2, wherein the nucleotide at position 14 on the antisense strand forms a wobble base pair with the target nucleotide.

6. The nucleic acid of claim 4 or 5, wherein the nucleotide at position 14 on the antisense strand comprises either inosine or uridine.

7. The nucleic acid of any one of claims 4-6, wherein the wobble base pair is I:C or U:G.

8. The nucleic acid of any one of claims 4-7, wherein:
the nucleotide at position 14 on the antisense strand comprises inosine if the target nucleotide comprises cytidine; or
the nucleotide at position 14 on the antisense strand comprises uridine if the target nucleotide comprises guanosine.

9. The nucleic acid of any one of claims 1-8, wherein the antisense strand comprises at least one modified nucleotide and/or at least one modified internucleotide linkage.

10. The nucleic acid of any one of claims 1-9, wherein the antisense strand comprises one or more nucleoside modifications selected from 2′-aminoethyl, 2′-fluoro, 2′-O-methyl, 2′-O-methoxyethyl, and 2′-deoxy-2′-fluoro-β-d-arabinonucleic acid.

11. The nucleic acid of any one of claims 1-10, wherein the antisense strand comprises at least one phosphorothioate internucleotide linkage.

12. The nucleic acid of any one of claims 1-11, wherein the antisense strand is 15 to 25 nucleotides in length.

13. The nucleic acid of any one of claims 1-12, wherein the antisense strand is 19 to 25 nucleotides in length.

14. The nucleic acid of any one of claims 1-13, wherein the antisense strand is 21 nucleotides in length.

15. The nucleic acid of any one of claims 1-14, wherein the sequence of the antisense strand is at least 80% identical to a nucleotide sequence of Table 1.

16. The nucleic acid of any one of claims 1-15, further comprising a sense strand of 15 to 40 nucleotides in length, wherein the sense strand forms a duplex region with the antisense strand.

17. The nucleic acid of claim 16, wherein the duplex region comprises a canonical or non-canonical base pairing between a nucleotide on the sense strand and the nucleotide at position 14 on the antisense strand.

18. The nucleic acid of claim 17, wherein:
the nucleotide on the sense strand comprises cytidine, adenosine, or uridine, if the nucleotide at position 14 on the antisense strand comprises inosine; or
the nucleotide on the sense strand comprises adenosine if the nucleotide at position 14 on the antisense strand comprises uridine.

19. The nucleic acid of any one of claims 16-18, wherein the sense strand comprises at least one modified nucleotide and/or at least one modified internucleotide linkage.

20. The nucleic acid of any one of claims 16-19, wherein the sense strand comprises one or more nucleoside modifications selected from 2′-aminoethyl, 2′-fluoro, 2′-O-methyl, 2′-O-methoxyethyl, and 2′-deoxy-2′-fluoro-β-d-arabinonucleic acid.

21. The nucleic acid of any one of claims 16-20, wherein the sense strand comprises at least one phosphorothioate internucleotide linkage.

22. The nucleic acid of any one of claims 16-21, wherein the sense strand is conjugated to at least one N-acetylgalactosamine (GalNAc) moiety.

23. The nucleic acid of any one of claims 1-22, wherein the sequence of the antisense strand, with the exception of the nucleotide that forms the wobble base pair, is 100% complementary to the contiguous sequence of the target mRNA.

24. A nucleic acid for reducing expression of a target mRNA, the nucleic acid comprising an antisense strand of Formula (I):
5′-X⁰¹X⁰²X⁰³X⁰⁴X⁰⁵X⁰⁶X⁰⁷X⁰⁸X⁰⁹X¹⁰X¹¹X¹²X¹³X¹⁴(X^{Y})_{b}Nₐ-3′ (I),
wherein:
each instance of N and X^{Y} is independently any type of nucleotide;
a is an integer from 0-2, inclusive;
b is an integer from 1-17, inclusive;
X⁰¹-X¹³ are each independently any type of nucleotide, with the proviso that X⁰¹—(X^{Y})_{b} is at least 90% complementary to a contiguous nucleotide sequence of the target mRNA; and
X¹⁴ is an abasic site or a nucleotide that does not form a canonical base pair with a target nucleotide at a corresponding position on the contiguous nucleotide sequence of the target mRNA.

25. The nucleic acid of claim 24, wherein the target nucleotide comprises either cytidine or guanosine.

26. The nucleic acid of claim 24 or 25, wherein X¹⁴ and the target nucleotide comprise a mismatched base pair.

27. The nucleic acid of claim 26, wherein the mismatched base pair is a wobble base pair.

28. The nucleic acid of claim 24 or 25, wherein X¹⁴ is a nucleotide that forms a wobble base pair with the target nucleotide.

29. The nucleic acid of claim 27 or 28, wherein X¹⁴ comprises either inosine or uridine.

30. The nucleic acid of any one of claims 27-29, wherein the wobble base pair is I:C or U:G.

31. The nucleic acid of any one of claims 27-30, wherein:
X¹⁴ comprises inosine if the target nucleotide comprises cytidine; or
X¹⁴ comprises uridine if the target nucleotide comprises guanosine.

32. The nucleic acid of any one of claims 24-31, wherein the antisense strand comprises at least one modified nucleotide and/or at least one modified internucleotide linkage.

33. The nucleic acid of any one of claims 24-32, wherein the antisense strand comprises one or more nucleoside modifications selected from 2′-aminoethyl, 2′-fluoro, 2′-O-methyl, 2′-O-methoxyethyl, and 2′-deoxy-2′-fluoro-β-d-arabinonucleic acid.

34. The nucleic acid of any one of claims 24-33, wherein the antisense strand comprises at least one phosphorothioate internucleotide linkage.

35. The nucleic acid of any one of claims 24-34, wherein a is 0.

36. The nucleic acid of any one of claims 24-35, wherein b is an integer from 1-11, inclusive.

37. The nucleic acid of any one of claims 24-36, wherein b is an integer from 5-11, inclusive.

38. The nucleic acid of any one of claims 24-37, wherein b is 7.

39. The nucleic acid of any one of claims 24-38, wherein the sequence of X⁰¹—(X^{Y})_{b} is at least 80% identical to a nucleotide sequence of Table 1.

40. The nucleic acid of any one of claims 24-39, further comprising a sense strand of 15 to 40 nucleotides in length, wherein the sense strand forms a duplex region with the antisense strand.

41. The nucleic acid of claim 40, wherein the duplex region comprises a canonical or non-canonical base pairing between a nucleotide on the sense strand and X¹⁴.

42. The nucleic acid of claim 41, wherein:
the nucleotide on the sense strand comprises cytidine, adenosine, or uridine, if X¹⁴ comprises inosine; or
the nucleotide on the sense strand comprises adenosine if X¹⁴ comprises uridine.

43. The nucleic acid of any one of claims 40-42, wherein the duplex region excludes each instance of N.

44. The nucleic acid of any one of claims 40-43, wherein the sense strand comprises at least one modified nucleotide and/or at least one modified internucleotide linkage.

45. The nucleic acid of any one of claims 40-44, wherein the sense strand comprises one or more nucleoside modifications selected from 2′-aminoethyl, 2′-fluoro, 2′-O-methyl, 2′-O-methoxyethyl, and 2′-deoxy-2′-fluoro-β-d-arabinonucleic acid.

46. The nucleic acid of any one of claims 40-45, wherein the sense strand comprises at least one phosphorothioate internucleotide linkage.

47. The nucleic acid of any one of claims 40-46, wherein the sense strand is conjugated to at least one N-acetylgalactosamine (GalNAc) moiety.

48. The nucleic acid of any one of claims 24-47, wherein the antisense strand is of Formula (II):
5′-X⁰¹X⁰²X⁰³X⁰⁴X⁰⁵X⁰⁶X⁰⁷X⁰⁸X⁰⁹X¹⁰X¹¹X¹²X¹³X¹⁴X¹⁵X¹⁶X¹⁷X¹⁸X¹⁹X²⁰X²¹-3′ (II),
wherein:
X⁰¹-X¹³ and X¹⁵-X²¹ are each independently any type of nucleotide, with the proviso that X⁰¹-X²¹ is at least 90% complementary to the contiguous nucleotide sequence of the target mRNA; and
X¹⁴ is a nucleotide comprising inosine or uridine.

49. A composition comprising a nucleic acid of any one of the preceding claims and a counterion.

50. A composition comprising a nucleic acid of any one of the preceding claims and a pharmaceutically acceptable carrier.

51. A method of reducing expression of a target mRNA in a cell, the method comprising contacting the cell with a nucleic acid or a composition of any one of claims 1 to 50.

52. The method of claim 51, wherein the cell is a mammalian cell.

53. The method of claim 51 or 52, wherein the cell is in vivo.

54. The method of claim 51 or 52, wherein the cell is in vitro.
